(19) United States
(12) Patent Application Publication
(10) Pub. No.: US 2026/0248549 A1
(43) Pub. Date: Aug. 27, 2026

(54) RESECTOSCOPE DEVICE, RESECTION TOOL AND RESECTOSCOPE

(71) Applicant: KARL STORZ SE & Co. KG, Tuttlingen (DE)

(72) Inventors: Peter SCHWARZ, Tuttlingen (DE); Christian GRAF, Tuttlingen (DE)

(73) Assignee: KARL STORZ SE & Co. KG, Tuttlingen (DE)

(21) Appl. No.: 19/545,841

(22) Filed: Feb. 20, 2026

(30) Foreign Application Priority Data
Feb. 24, 2025 (DE) 102025106809.7

(51) Int. Cl. A61B 18/14; A61B 1/00; A61B 1/015; A61B 1/05; A61B 1/307; A61B 18/00

(52) U.S. Cl. CPC A61B 18/14; A61B 1/00066; A61B 1/00135; A61B 1/00181; A61B 1/015; A61B 1/05; A61B 1/307; A61B 2018/00172; A61B 2018/00184; A61B 2018/00517; A61B 2018/00601; A61B 2018/0091; A61B 2018/00982; A61B 2018/1407; A61B 2218/002

(57) ABSTRACT

The present disclosure relates to a resectoscope device comprising a shaft; an image acquisition device, which is located on a distal end piece of the shaft and defines an observation region; a resection tool with a tool tip and a carrier arm ; and a joint by means of which the carrier arm is bendable in an articulated manner, wherein, in at least one angular position of the carrier arm, a distance between the tool tip and a longitudinal axis of the distal end piece exceeds a radius of a cross-sectional profile of the distal end piece by at least a factor of 2.

### CROSS-REFERENCE TO RELATED APPLICATIONS

This application claims priority of German Patent Application No. DE 102025106809.7 on February 24, 2025, the contents of which are incorporated herein.

### TECHNICAL FIELD

The present disclosure relates to resectoscope devices, a resection tool, and a resectoscope.

### BACKGROUND

A resection is the surgical removal of tissue from an organ or a tumor. Depending upon the objective and anatomical circumstances, different types of resections can be performed. In cases of difficult-to-access tissue, surgical removal can be performed endoscopically using a resectoscope. Such a resectoscope is used, for example, in transurethral resection, in which diseased tissue is removed from the bladder or prostate. The operation is performed endoscopically through the urethra of the patient, without the need for an incision.

A conventional resectoscope for transurethral resection comprises a shaft, at the distal end portion of which an observation optics, a rinsing device, and a resection tool are arranged. The shaft allows access to the bladder through the urethra and provides stability during the procedure. The distal end portion of the shaft can therefore be positioned during the procedure by sliding the shaft back and forth along the urethra and by pivoting the shaft in the bladder. During such pivoting, a portion, proximal to a pivot point, of the shaft is moved by a user within a large manipulation cone. This is necessary in order to be able to move a portion of the shaft distal to the pivot point within a sufficiently large application cone. A proximal portion of the shaft is coupled to an operating module that is held and moved by a user during the procedure. The operating module also comprises an actuating device by means of which the resection tool can be moved relative to the distal end portion.

A resection using such a conventional resectoscope is usually performed as follows. The user grasps the resectoscope at the operating module. He then pushes the shaft through the urethra of the patient in such a way that the distal end portion of the shaft protrudes into the bladder. The bladder is then filled using the rinsing device.

Then, an imaging device is activated, and the user is provided with an overview of the interior of the bladder by endoscopic observation. In this way, he can identify the tissue sites to be removed within the bladder. To remove them, the user pushes the shaft forwards and pivots it as needed until the distal end portion is near the affected tissue site. The user then orients the observation optics of the imaging device so that the tissue site is clearly visible in the field of view. To ablate the tissue, the user now operates the actuating device, thereby pushing the resection tool back and forth. In an iterative process, the tissue can be ablated layer by layer by actuating the actuating device. To reach a larger tissue region with the resection tool, which is regularly necessary, the user also pivots and shifts the shaft.

In the manner described, transurethral resection can be used primarily for the treatment of superficial bladder cancer by resection in a urinary bladder. The inventors of the present disclosure have recognized that this is difficult for the user to perform and that complications may arise. During the resection, the user must, according to the prior art, strongly pivot the resectoscope and move it intensively along the urethra. This can impair the precision of tissue ablation. Furthermore, complications can occur for the patient, wherein the pivoting of the shaft in particular often leads to postoperative discomfort for the patient. For example, injuries to muscles and tissue that are not directly visible or severe irritation of the mucous membranes can occur during the procedure. These complications can subsequently lead to urinary incontinence. As mentioned, using a described conventional resectoscope always requires extensive pivoting of the shaft during the resection. The resection is performed in the form of a rowing movement by means of a combination of pivoting and pushing movements. This puts particular strain on the bladder neck.

Based upon the prior art, the disclosure is based upon the object of providing a resectoscope by means of which a resection can be carried out precisely and/or gently for a patient.

The object is achieved according to the disclosure by resectoscope devices, a resectoscope tool, and a resectoscope as are described herein and defined in the claims.

According to one aspect, the present disclosure provides a resectoscope device, in particular for the transurethral resection of a tissue. The resectoscope device comprises a shaft and an image acquisition device, which is located on a distal end piece of the shaft and defines an observation region. Furthermore, the resectoscope device comprises a resection tool with a tool tip and a carrier arm, as well as a joint by means of which the carrier arm is bendable in an articulated manner. In at least one angular position of the carrier arm, a distance between the tool tip and a longitudinal axis of the distal end piece exceeds a radius of a cross-sectional profile of the distal end piece by at least a factor of 2.

According to a further aspect, the present disclosure provides a resectoscope device, in particular for the transurethral resection of a tissue. The resectoscope device can in particular have the features mentioned above. It comprises a shaft and an image acquisition device, which is located on a distal end piece of the shaft and defines an observation region. Furthermore, the resectoscope device comprises a resection tool with a tool tip and a carrier arm, as well as a joint by means of which the carrier arm is bendable in an articulated manner. In this case, the shaft defines a manipulation cone within which a portion, proximal to a pivot point, of the shaft can be manipulated by a user. The tool tip can be moved by manipulating the proximal portion of the shaft within the manipulation cone and selecting at least one, in particular exactly one, angular position of the carrier arm on a surface portion, located within the observation region, of an application cone. The aperture angle of the application cone is at least 1.5 times larger than the aperture angle of the manipulation cone.

According to a further aspect, the present disclosure provides a resectoscope device, in particular for the transurethral resection of a tissue. The resectoscope device can in particular have the features mentioned above. It comprises a shaft and an image acquisition device, which is located on a distal end piece of the shaft and defines an observation region. Furthermore, the resectoscope device comprises a resection tool with a tool tip and a carrier arm, as well as a joint by means of which the carrier arm is bendable in an articulated manner. In this case, the image acquisition device comprises at least two image acquisition units that are directed in different viewing directions, wherein the image acquisition units each define an observation sub-region of the observation region. The tool tip is movable between the observation sub-regions by bending the carrier arm.

Furthermore, the present disclosure provides a resection tool, in particular for an inventive resectoscope device. It comprises a tool tip, a carrier arm, and a joint by means of which the carrier arm is bendable in an articulated manner. The resection tool defines a bending portion that is bendable by means of the joint, wherein the length of the bending portion is between 15 mm and 50 mm and in particular between 25 mm and 35 mm.

Furthermore, the present disclosure provides a resectoscope having an inventive resectoscope device.

The features according to the disclosure allow for a precise and patient-friendly transurethral resection. Patient comfort and patient safety are increased, and the risk of complications such as urinary incontinence is reduced. This, in turn, increases the acceptance of transurethral resections, thereby reducing the consequences of long-postponed resections. In particular, the extensive pivoting movements necessary after conventional resection can be reduced by the features according to the disclosure. A fundamentally different way of performing the resection is thereby made possible, which is gentle on the patient and delivers high precision.

For example, after inserting the distal end piece, a user can bend a portion of the carrier arm once by means of the joint. This allows the tool tip to be guided into the vicinity of tissue to be removed, such as a tumor. The user therefore no longer needs to move the entire resectoscope in a large manipulation cone or pivot the proximal portion of the shaft to a great extent outside the bladder in order to reach the tissue to be removed. As a result, the urethra is subjected to less extensive movements. This is possible specifically because the distance of the tool tip, which is large relative to the radius of the distal end piece, can be adjusted by bending the carrier arm. Once set, the tool tip remains in this angular position at least temporarily while the tissue ablation is carried out. For this, the user must now move the resection tool and in some cases the resectoscope back and forth, making only small pivoting movements. With a conventional resectoscope, the user would have to pivot the shaft to a great extent and move it on a manipulation cone with a large aperture angle, as described above. In other words, the conventional rowing movement during resection can be partially or even largely avoided by means of the inventive features.

The application cone, within which tissue ablation is possible, therefore has a larger aperture angle than the manipulation cone. Accordingly, for example, tissue portions on the lower or upper bladder wall are easily accessible, even though the manipulation cone can be kept small. The large application cone can be achieved by bending by means of the joint. Once the angular position is selected, the handling of the inventive resectoscope is comparable with conventional resectoscopes, allowing users to draw on their experience with conventional resectoscopes when performing procedures. Tissue ablation is performed by moving the resection tool and/or the shaft back and forth. In contrast, comparatively small pivoting movements of the shaft are now necessary.

An embodiment is likewise conceivable in which the tissue ablation is carried out by continuously bending the portion, bendable by means of the joint, of the carrier arm relative to the distal end piece. The angular position of the carrier arm is changed continuously, and a kind of scooping movement is executed. As a result, the tool tip is guided along the tissue to be ablated, and the tissue is ablated layer by layer. Accordingly, the necessary forward and backward movement of the resection tool and/or the shaft can also be reduced, since the ablation movement is already carried out by bending the carrier arm.

Furthermore, by using the at least two image acquisition units, a large observation region can be provided that can cover large parts of the inner wall of the bladder (at least on one side). This can extend as far as the bladder inlet or bladder neck. The bladder inlet can therefore be viewed with at most slight pivoting of the shaft in the manipulation cone. Since the tool tip is movable between the observation sub-regions, it can be observed continuously by means of the image acquisition device during bending and moved into the vicinity of the bladder inlet. Thus, a large portion of the application cone can be covered by means of the image acquisition device, meaning that the user does not have to pivot the distal end piece extensively by manipulating the shaft within the manipulation cone for endoscopic imaging. Even without extensive pivoting, the resection, e.g., on the tissue portions on the lower or upper bladder wall, can be monitored by means of endoscopic imaging.

A "resectoscope" can be understood as a medical instrument for the endoscopic removal of tissue within a hollow organ, in particular the bladder and/or the prostate. A resectoscope can allow minimally invasive procedures, in particular through controlled resection movements and/or endoscopic imaging.

The term “resectoscope device” is to be understood as meaning, in particular, a preferably functional component, in particular a subassembly and/or a structural and/or functional component of a resectoscope. Preferably, the resectoscope device can form the resectoscope at least partially, preferably at least to a large extent, and particularly preferably completely. For example, the resectoscope device can be configured to be inserted at least partially and preferably at least to a large extent into a cavity or into, in particular, an artificial and/or natural cavity, in particular a body cavity, in particular to inspect it and to modify parts thereof, in particular to remove it. The resectoscope device can be a medical resectoscope device.

In the context of this disclosure, “configured” can be understood to mean, in particular, programmed, formed, designed, and/or equipped. The fact that a component is configured for a specific function can be understood within the context of this disclosure in particular to mean that the component fulfills and/or executes this specific function in at least one application and/or operating state.

“Distal” is to be understood to mean, in particular, facing a patient during operation and/or facing away from an operator and/or user during operation. In particular, proximal is the opposite of distal. “Proximal” is to be understood to mean, in particular, facing away from a patient and/or facing an operator and/or user during operation.

The shaft can, for example, comprise an elongated, cylindrical, in particular hollow-cylindrical element that carries instruments, devices, and/or the like, by means of which a resection can be performed. It can allow the insertion of the instruments, devices, and/or the like into the cavity, in particular the bladder. In general, access to the cavity can be achieved by means of the shaft. In the process, the shaft can be held and/or manipulated by a user at its proximal portion to orient the instruments, devices, and/or the like within the cavity. By means of the manipulation, the resection can in particular also be performed by the user. The shaft can be correspondingly rigid and/or inflexible. The shaft can have an outer diameter of, for example, up to 15 mm, e.g., up to 10 mm, in particular up to 9 mm, preferably up to 8 mm, and a length of, for example, 10 cm to 50 cm, in particular 15 cm to 40 cm, preferably 20 cm to 30 cm.

In particular, the shaft can be inserted into the patient's cavity such that the distal end piece is positioned within the cavity. The distal end piece can carry functional units of the resectoscope device, by means of which the resection can be performed. The distal end piece can define a distal end portion of the shaft and extend over, for example, up to 5 cm, in particular up to 4 cm, preferably up to 3 cm, of the shaft.

An “elongated part” is to be understood in particular as a component whose main extension is at least a factor of five, preferably at least a factor of ten, and particularly preferably at least a factor of twenty, larger than a largest extension of the component perpendicular to its main extension, i.e., in particular a diameter of the component. A “main extension” of a component should be understood in particular as its longest extension along its main extension direction. A “main extension direction” of a component is to be understood to mean, in particular, a direction extending parallel to a longest edge of a smallest imaginary cuboid which only just fully encloses the component and which preferably passes through a geometric center and/or a center of mass of the component.

Endoscopic imaging can be carried out by means of the image acquisition device. The image acquisition device can be configured to image objects within the observation region, in particular the interior of the cavity, and/or to generate image data. An object can, for example, comprise tissue, in particular a portion of the bladder wall. The image data can be used to generate a representation of the interior of the cavity on a display device. The display device allows the user to observe the inside of the bladder and the progress of the resection or ablation of tissue. The image acquisition device can be positioned, in particular by the user, within the cavity, in particular independently of the resection tool.

The image acquisition device can comprise at least one image acquisition unit, which may be designed in particular by an input optics and/or an image sensor for image generation, in particular a CCD chip or CMOS chip. The input optics can be arranged, for example, at a distal end face of the shaft.

By means of the image acquisition device with the at least two image acquisition units directed in different viewing directions, a large observation region for endoscopic imaging can be achieved. Each of the image acquisition units can be formed at least by an input optics and/or an image sensor for image generation, in particular a CCD chip or CMOS chip.

The at least two image acquisition units can in particular comprise a first image acquisition unit and a second image acquisition unit, wherein the first image acquisition unit is directed in a distal viewing direction, and wherein the second image acquisition unit is directed in a proximal viewing direction. The viewing direction from two different sides further improves the image available to the user through the images taken from different perspectives, and in particular improves visibility when setting a starting point for tissue removal.

In some embodiments, the image acquisition device comprises exactly two, exactly three, and/or more or at least two image acquisition units directed in different viewing directions, the image acquisition units together defining a continuous observation region. The observation sub-regions together define the observation region and can in particular overlap.

The viewing direction can be directed in a proximal direction with respect to the shaft. The viewing direction can be directed distally with respect to the shaft. The viewing direction can be defined by a viewing angle relative to the longitudinal axis of the shaft. A viewing angle of 0° to less than 90° can be interpreted as directed in a distal viewing direction, and a viewing angle of more than 90° to 180° can be interpreted as directed in a proximal direction.

The image acquisition device can comprise at least three image acquisition units directed in three different viewing directions. The arrangement of multiple image acquisition units with different viewing directions improves the imaging of tissue to be removed that is available to a user. The at least three image acquisition units can define three different observation sub-regions and in particular one observation sub-region each, wherein the different observation sub-regions can overlap at least partially. The observation sub-regions can therefore each have a portion that lies within an adjacent observation sub-region. With overlapping observation sub-regions viewed from different viewing directions, the imaging, available to the user, of the tissue to be removed is further improved.

The overlapping observation sub-regions can jointly define the observation region. The multiple image acquisition units can be arranged offset in a longitudinal direction and/or with respect to a longitudinal axis of the shaft, in particular the distal end piece of the shaft, and in particular arranged one behind the other.

The one or more image acquisition unit(s) can each be formed by camera modules, in particular camera modules having short-profile lenses or so-called camera cubes with wafer-level optics. The one or more image acquisition units each have, in particular, an individual viewing angle. The individual viewing angle can, for example, be 60°, 65°, 70°, 75°, 80°, 85°, 90°, 95°, 100°, 105°, or 110°.

The viewing direction of an image acquisition unit can be a primary viewing direction of the relevant image acquisition unit. For example, the viewing direction can be a central axis of an input optics of the relevant image acquisition unit. The individual viewing angle can be an angle that is spanned relative to the viewing direction. In particular, the central axis can be an angle bisector of the individual viewing angle.

Depending upon the design of the image acquisition units, this can be defined by two individual viewing angles that are perpendicular to each other. These can, for example, be defined as viewing angles relative to a transverse axis or viewing angles relative to a vertical axis, each perpendicular to the central axis and/or the viewing direction. In particular in cases where an image acquisition unit comprises a rectangular image sensor, both viewing angles belonging to the image acquisition unit can be assigned to a short and a long side of the rectangular image sensor.

The specification of an individual viewing angle can also be understood as the largest viewing angle of the relevant image acquisition unit and/or as a viewing angle that corresponds to a diagonal of the relevant image sensor. An image acquisition unit can also be described by a horizontal viewing angle, a vertical viewing angle, and a diagonal viewing angle. Specifications relating to a viewing angle can refer to any of these three variables and preferably refer to a diagonal viewing angle.

To illuminate the interior of the cavity, the imaging regions and/or the observation region, an illumination device may be arranged at a distal end piece, configured to provide illumination light, of the shaft. Illuminating the imaging region and/or the observation region supports the capture of an image of it with the image acquisition device and thus in particular increases the image quality of an image provided to the user. The illumination device may comprise a plurality of illumination units which are directed in particular in different illumination directions. The individual illumination units can be formed by light-emitting diodes and/or laser diodes and/or light guides.

A compact resectoscope device can be provided if the image acquisition device is integrally designed with the illumination device. In particular the distal end piece can be designed to be compact and utilize installation space efficiently. In some embodiments, the image acquisition device and the illumination device can be designed in pairs. This can mean that each illumination unit is integrally designed with an image acquisition unit.

The resection tool can be configured to remove a tissue by cutting and/or vaporization. The resection tool can contain an electrically conductive material, in particular a wire, via which an electric current can flow. The resection tool can be designed as an HF tool (high-frequency tool). The resection tool can have a resection loop. The resection tool can be loop-shaped, hook-shaped, or spherical.

The resection tool may, in particular, comprise an HF loop (high-frequency loop). Alternatively or additionally, the resection tool may comprise a laser for cutting and/or vaporization. The laser can be designed in particular for holmium laser enucleation or KTP laser vaporization, or can be configured as a continuous-wave laser. These laser methods differ in the wavelength and energy of the laser light used, and in particular in their energy effect in tissue and their penetration depth. Alternatively or additionally, the resection tool can have a monopolar or a bipolar HF tool (high-frequency tool). The monopolar HF tool can be designed as a hook electrode, ball electrode, or spatula electrode for cutting and/or vaporization. The bipolar HF tool can be designed in particular as bipolar tweezers or forceps for cutting and/or vaporization. The resection tool can also be configured to perform contact coagulation on tissue. Depending upon the application, one of the described designs of the resection tool may be particularly suitable.

The tool tip can define the part of the resection tool that comes into contact with the tissue to be removed. Furthermore, the tool tip can, for example, comprise a supply line portion and/or the like, which extends further distally at least from a distal end of the carrier arm. In particular, the tool tip can be positioned distally to the carrier arm and/or distally adjoin the carrier arm.

A carrier arm can be configured to connect the tool tip to a proximal module, in particular an operating module. If the tool tip is electronically operated, an electrical supply line, e.g., a cable, can be guided within the carrier arm, and/or the carrier arm can form such a supply line.

In some embodiments, the carrier arm can be shaped approximately like a tube and accommodate at least one further functional unit and/or further supply lines. Furthermore, a carrier arm can be designed to be rigid, inflexible, and/or mechanically stable. If, for instance, a resection is performed with a single selection of an angular position of the carrier arm, as described at the beginning, the carrier arm can be designed to be so rigid, inflexible, and/or mechanically stable that the user can apply a force suitable for tissue ablation to the tissue to be removed without the carrier arm deforming significantly.

In particular a distal portion of the carrier arm is bendable by means of the joint. This can mean that the angular position of a portion located distally to the joint can be changed relative to a portion located proximally to the joint. The joint can in particular be located on a longitudinal axis of the carrier arm and/or be integrated into the carrier arm. In some embodiments, the carrier arm at least partially forms the joint. By bending the carrier arm, the tool tip can be positioned within the patient's cavity, in particular without manipulation of the proximal portion of the shaft.

The bendable portion of the carrier arm can be part of a "bending portion." In other words, the resection tool can define the bending portion, wherein the bending portion comprises a portion of the carrier arm and/or at least part of the tool tip. The bending portion is bendable by means of the joint. The bending portion can comprise the tool tip and a portion, in particular a distal portion, in particular an end portion.

The term "bendable in an articulated manner" can be understood in particular to mean that one part of the carrier arm is mechanically adjustable or rotatable in a targeted manner relative to another part of the carrier arm. "Bending in an articulated manner" can comprise a targeted movement and/or orientation. A component can be understood as "articulated" if it is connected to another component via a mechanical joint, in particular such that a relative movement of the two components toward each other is possible. This can comprise uniaxial mobility - for example, a tilting or pivoting movement around a fulcrum. "Bendable in an articulated manner" can also comprise multi-axial and/or multidirectional mobility, which is made possible, for example, by means of a ball joint. The articulated bendability can be achieved without tools by friction, a locking mechanism, and/or a snap system. Additionally, in some embodiments, an arrester can be provided by means of which the bendable portion of the carrier arm can be fixed in an angular position.

The carrier arm can in particular define multiple angular positions. In some embodiments, the carrier arm can be bendable continuously by means of the joint. In other embodiments, the carrier arm can be bendable stepwise by means of the joint. An angle of the angular position can be defined in relation to a longitudinal axis of the shaft, in particular the distal end piece and/or a portion of the carrier arm located, in particular immediately, proximally to the joint. In an insertion configuration in which the resectoscope device can be inserted into the cavity, an insertion angular position can be defined, wherein the bending portion and the longitudinal axis of the distal end piece and/or of the portion of the carrier arm located, in particular immediately, proximally to the joint, form an angle of, for example, between 5° and -5° inclusive, in particular between 3° and -3° inclusive, preferably between 1° and -1° inclusive. Basically, the insertion angular position can mean a zero angular position in which the bending portion is arranged parallel to the longitudinal axis of the distal end piece. The angle is measured, for example, starting from the insertion angular position or a coaxial arrangement of the bending portion and one of the aforementioned longitudinal axes.

The distance between the tool tip and the longitudinal axis of the distal end piece can mean in particular a distance between the most distal point of the tool tip, in particular in the insertion angular position. The distance can also mean a minimum distance between a point on the tool tip furthest from the longitudinal axis and the longitudinal axis, which can be adjusted in particular by the bending by means of the joint.

This minimum distance can exceed the radius of the cross-sectional profile of the distal end piece by at least a factor of 2, in particular by at least a factor of 2.5, 3, 3.5, 4, 4.5, and/or greater than 4.5. In a view of the resectoscope device from the distal side, the tool tip can therefore project beyond the cross-section of the distal end piece by at least this factor, in particular if the longitudinal axis is on a geometric center and/or a center of mass of the cross-section of the distal end piece. The radius of the cross-sectional profile can mean the largest radius of the cross-sectional profile. In other words, the radius that serves as a reference value can be determined at the point along the longitudinal axis of the distal end piece where the cross-sectional profile has its greatest spatial extent, in particular transverse to the longitudinal axis. In some embodiments, the distal end portion can have a smaller radius of the cross-sectional profile than a more proximal portion of the shaft. In such a case, e.g., in at least one angular position of the carrier arm, the distance between the tool tip and the longitudinal axis of the distal end piece can exceed a radius of a cross-sectional profile of the shaft by at least a factor of 2.

In particular in an application configuration, the distance between the tool tip and the longitudinal axis of the distal end piece exceeds a radius of a cross-sectional profile of the distal end piece by at least a factor of 2 in at least one angular position of the carrier arm. The resection can be performed in the application configuration. Therefore, in the insertion configuration, the resection device can be inserted into the cavity, and, in the application configuration, the resection or tissue ablation can be carried out.

In a state inserted into the cavity, the shaft can be rotatable about a pivot point and/or linearly displaceable through the pivot point. This pivot point can be understood as a so-called trocar point.

For example, in the inserted state, the shaft extends partially outside the cavity and partially inside the cavity. In particular, the proximal portion of the shaft is located outside the cavity, and at least the distal end piece is located inside the cavity.

The shaft, in particular the proximal portion of the shaft, can be moved by the user in a conical region about the pivot point or trocar point. The pivot point or trocar point describes the apex of the cone. The movement or manipulation can comprise pivoting and moving the shaft back and forth.

For each aperture angle selected by pivoting the proximal portion of the shaft, the user can therefore trace a lateral surface of an imaginary, extracavitary cone. This can accordingly be defined as a manipulation cone. The imaginary, extracavitary cone can in particular have the shape of a right circular cone.

A single manipulation cone therefore has a specific aperture angle. The user can manipulate the proximal shaft portion within the manipulation cone by pivoting and linearly moving it through the pivot point, wherein the aperture angle is not exceeded.

The distal end piece and the proximal portion of the shaft can both be located one behind the other on the longitudinal axis of the shaft. Accordingly, the distal end piece describes a movement within a movement cone when the user manipulates the proximal portion of the shaft within the manipulation cone. The movement cone then has an aperture angle that corresponds to the aperture angle of the manipulation cone. Accordingly, the movement cone can correspond to a mirror image of the manipulation cone. In the insertion configuration, the tool tip can therefore be movable substantially within the movement cone by manipulating the proximal portion of the shaft.

Additionally, the user can bend the carrier arm by means of the joint. More precisely, the user can bend the bending portion. The user can thereby select an angular position of the carrier arm. In the angular position, the tool tip can be at a distance from the longitudinal axis of the distal portion. In particular in the application configuration, the carrier arm is bent and/or can have an angular position, in particular one that differs from the angular position of the insertion configuration.

In the application configuration, the tool tip can be moved outside the movement cone by manipulating the proximal portion of the shaft, in particular due to the distance of the tool tip from the longitudinal axis of the distal end piece.

In the bent state and/or in the application configuration, the tool tip can be moved on the application cone by manipulating the proximal portion of the shaft within the manipulation cone. A certain manipulation cone then allows the tool tip to move on a certain application cone.

Since the carrier arm can be bent and, in particular, the tool tip be at a distance from the longitudinal axis of the distal end piece, the application cone can have a larger aperture angle than the movement cone and, accordingly, the manipulation cone. If the proximal portion of the shaft is moved within a specific manipulation cone, the tool tip can be movable on the surface of the application cone. A surface portion of the application cone, which can, for example, correspond to the lateral surface of a truncated cone, can be traced with the tool tip. The pivot point itself can be excluded from the surface of the application cone that can be traced with the tool tip by manipulating the proximal portion of the shaft.

To move the tool tip on the lateral surface of the truncated cone and/or the surface portion of the application cone, the proximal portion of the shaft can be moved closer to the axis of the manipulation cone within the manipulation cone. The surface of the application cone can therefore be traced by a linear movement through the pivot point and a pivoting movement about the pivot point of the proximal portion of the shaft within the manipulation cone by means of the tool tip. Accordingly, the tool tip can be moved within the application cone by manipulating the proximal portion of the shaft within the manipulation cone, wherein the application cone has a larger aperture angle than the manipulation cone.

The application cone can generally specify the boundary of a region within which a resection can be performed while maintaining a specific manipulation cone. Due to the bendability of the carrier arm, the manipulation cone can have a comparatively small aperture angle. As a result, a resection that is gentle on the patient can be performed. This can also be comfortable for the user to perform, since the user can easily and efficiently reach remote and/or distant tissue regions within the bladder.

In other words, due to the choice of angular position, a larger region can be reached by means of the tool tip with the same degree of pivoting about the pivot point of the shaft, in comparison with a conventional resectoscope device that has no joint or no joint with the inventive features. In particular, the carrier arm is bendable by means of the joint at least such that the aperture angle of the application cone is at least 1.5 times, in particular 2.0 times, 2.5 times, 3.0 times, 3.5 times, 4.0 times, and/or 4.5 times, as large as the aperture angle of the manipulation cone.

For example, according to some embodiments, the aperture angle of the manipulation cone does not have to exceed an aperture angle of 15°, in particular 10°, in order to be able to reach at least substantially the entire bladder wall by means of the resection tip. The necessary aperture angle can depend upon the patient's anatomy as well as the length of the bending portion. If the length of the bending portion is between 15 mm and 50 mm, and in particular between 25 mm and 35 mm, at least substantially the entire bladder wall can be reached with the tool tip for tissue ablation with a small aperture angle of the manipulation cone. It is conceivable that the tool tip and/or the resection tool be selected in a patient-specific manner in order to match the length of the bending portion to the patient's anatomy.

In particular, the region that can be traced by the tool tip can intersect with the observation region. The overlapping part can be referred to as the surface portion of the application cone on which the tool tip can move. This is therefore located within the observation region. Accordingly, the tool tip can be observed using endoscopic imaging when tracing the surface portion of the application cone. The surface portion does not necessarily have to be delimited exclusively by the observation region. A boundary, in particular a boundary proximal to the pivot point, can be defined by the choice of angular position. A distal boundary can be delimited by the observation region.

According to some embodiments, the resection tool defines the bending portion that is bendable by means of the joint. The distance of the surface portion of the application cone from the pivot point can be at least twice the length of the bending portion, in particular when choosing exactly one angular position. This means that even tissue portions of the cavity, in particular the bladder, that are far away from the pivot point can be reached with the tool tip. The geometric relationships can generally refer to a specific angular position. It is possible, for example, to reach tissue portions that are at least 10 mm, in particular at least 20 mm, preferably at least 30 mm, and particularly preferably at least 40 mm, away from the bladder inlet. The distance can in each case mean a minimum distance, i.e., in particular a distance between a point of the bladder inlet closest to the tissue portion and a point of the tissue portion closest to the bladder inlet.

In principle, the length of the bending portion can comprise the length of the tool tip and the length of a portion, bendable by means of the joint, of the carrier arm.

The phrase "movable between the observation sub-regions" can also be understood to mean that the tool tip is movable from one observation sub-region into another observation sub-region by bending the carrier arm. Alternatively, it could therefore be formulated, for example, that the tool tip is movable from a first observation sub-region of a first image acquisition unit into a second observation sub-region of a second image acquisition unit by bending the carrier arm. In particular, the tool tip is movable back and forth between the observation sub-regions. In particular, the tool tip is movable out of one of the observation sub-regions, in particular the first observation sub-region, by bending the carrier arm.

Furthermore, the carrier arm can be bendable relative to the distal end piece of the shaft by means of the joint, wherein in particular the distal end piece is immovable relative to a main body of the shaft. This allows for a gentle resection, in which the user needs to pivot the shaft at most slightly. Since the image acquisition device is arranged on the distal end piece, it remains stationary when the carrier arm is bent. Accordingly, the image acquisition device does not need to be moved during the transfer from the insertion configuration to the application configuration. The carrier arm can be bendable independently of the image acquisition device and/or the distal end piece. The main body can be a portion of the shaft that is located inside the ureter when in the state inserted into the bladder. Furthermore, the main body can, for example, comprise the proximal portion of the shaft. It can, for example, form a basic structural element of the shaft and provide in particular the fundamental shape and stability of the shaft. The main body can therefore represent the central carrier element to which at least the distal end piece is attached. The distal end piece can be inflexibly, rigidly, and/or immovably connected to the main body.

Gentle resection can be performed if the shaft and the resection tool can be moved independently of each other along a longitudinal axis of the shaft. Therefore, it is not necessary to move the entire resectoscope device back and forth to remove tissue. For example, the resection tool can be coupled to a proximal operating module, which, when operated by the user, moves the resection tool independently of the shaft along the longitudinal axis of the shaft.

Furthermore, the resection tool can define the bending portion that is bendable by means of the joint, wherein the length of the bending portion exceeds the radius of the cross-sectional profile of the distal end piece by at least a factor of 2, in particular by at least a factor of 3. For an understanding and the advantages of these features, reference is made to the explanations above.

Furthermore, the length of the bending portion can be between 15 mm and 50 mm, and in particular between 25 mm and 35 mm. For an understanding and the advantages of these features, reference is made to the explanations above.

According to some embodiments, the resectoscope device further comprises a rinsing device by means of which a region that can be treated by the resection tool can be rinsed. The rinsing device comprises a supply line by means of which a rinsing fluid can be conducted along the shaft, wherein the supply line extends within the carrier arm. During tissue ablation, the ablated tissue can be washed away, ensuring a good visibility of the tissue being treated. The rinsing device can comprise a channel that is incorporated into the carrier arm and/or extends along a longitudinal axis of the carrier arm. The channel can form the supply line. Alternatively, the supply line can comprise a tube that extends within a cavity of the carrier arm along the longitudinal axis from proximal to distal. This allows rinsing fluid to be introduced proximally into the supply line and conducted along the carrier arm into the cavity. The rinsing fluid can also be used to fill the bladder in order to stretch the bladder wall. In particular, in a distal portion of the carrier arm that is inserted into the bladder or cavity during a resection, the supply line can be arranged within the carrier arm.

Furthermore, the rinsing device can comprise an outlet that is arranged on a portion, bendable by means of the joint, of the carrier arm. In other words, the bending portion can comprise the outlet, and/or the outlet can be arranged on a portion of the bending portion that comprises the carrier arm. The outlet can therefore be bent by actuating the joint. When the carrier arm is bent, the outlet and the tool tip can move together. The outlet of the rinsing device can therefore have a fixed relative position in relation to the tool tip. This allows for efficient rinsing and/or removal of ablated tissue residues. The rinsing fluid can be delivered precisely regardless of the angular position.

According to some embodiments, the resection tool comprises a further bendable carrier arm. The tool tip can be arranged between the carrier arm and the further carrier arm. At least in an insertion configuration, in particular the shaft extends between the bendable portions of the carrier arms. A space-saving, compact resectoscope device can thereby be provided. The tool tip can in particular comprise an HF resection loop. A supply line can be arranged in each of the carrier arms, wherein each of the supply lines can be a pole.

Tissue portions to be removed can also be located in the longitudinal direction of the shaft on the bladder wall or opposite the bladder inlet. In order to be able to achieve this easily and with minimal pivoting of the proximal portion of the shaft, the tool tip can comprise an HF resection loop comprising a supply line portion and a working portion, wherein the working portion is bent relative to a longitudinal axis of the supply line portion by a loop angle of at most 90°. In particular, the working portion extends partially distally from the supply line portion. Conventional resectoscopes with an HF resection loop typically have loop angles greater than 90°. These must be pivoted to a corresponding extent so that the working portion can reach the tissue to be removed opposite the bladder inlet. The working portion can be the portion of the tool tip that comes into contact with the tissue to be removed for tissue ablation. The supply line portion can comprise a supply line, and/or a supply line can form at least part of the supply line portion. In some embodiments, the supply line portion has electrical insulation. A loop angle of 0° can mean that the working portion is arranged coaxially with the supply line portion and/or extends distally from the supply line portion on the longitudinal axis of the supply line portion. For example, with a loop angle of 45°, the working portion extends obliquely from the supply line portion partly distally and partly perpendicular to the longitudinal axis of the supply line portion.

Comprehensive endoscopic imaging of the bladder can be achieved if, for the purpose of performing a resection, the distal end piece of the shaft can be arranged at least partially within the bladder such that the bladder neck is within the observation region of the image acquisition device. Even complex resections near the bladder neck can be performed with slight pivoting of the resectoscope, wherein the tool tip can be kept within the observation region. In accordance with the above statements regarding the image acquisition device, to which reference is made, the image acquisition device can comprise at least two image acquisition units that are directed in different viewing directions, wherein the image acquisition units each define an observation sub-region of the observation region. One of the viewing directions can be arranged such that the observation sub-region of the associated image acquisition unit intersects the shaft.

Tissue of the entire bladder wall can be easily reached with the tool tip if the resection tool, together with the shaft, can be rotated about a longitudinal axis of the shaft. The image acquisition device can therefore be rotated together with the tool tip, and, for example, the roof of the bladder can be reached with the tool tip, wherein it can still be observed by means of endoscopic imaging.

The resectoscope device can also comprise an insertion sleeve that can be partially inserted into a cavity of the patient and defines a longitudinal axis. The shaft can extend within the insertion sleeve, and the shaft and the resection tool can be moved independently of each other relative to the insertion sleeve along the longitudinal axis of the insertion sleeve. In particular, the shaft and the resection tool can be inserted into the cavity together with the insertion sleeve. Thanks to the insertion sleeve, tissue damage and/or irritation caused by the linear movement of the shaft through the pivot point can be reduced.

The insertion sleeve can be designed as an elongated tube and/or comprise, in particular, such a tube along whose longitudinal axis a guide channel can run. In particular, it can be configured to provide access to a bladder of a patient. It can be positioned in and/or inserted into the urethra of the patient. In this arrangement, the longitudinal axis and, in particular, the guide channel can extend along the urethra. In this case, the insertion sleeve extends from a proximal side to a distal side. The shaft and/or the resection tool can be guided at least partially and/or portion by portion in the guide channel and, in particular, can be advanced in a guided manner. In some embodiments, the shaft can be guided in the insertion sleeve, and the resection tool can be guided on the shaft. The insertion sleeve can therefore accommodate the shaft and the resection tool, in particular on a portion that can be arranged within the urethra. Other modules can also be accommodated in the insertion sleeve - for example, a suction channel for suctioning rinsing fluid from the bladder. The insertion sleeve can be mounted on the shaft in a captive manner. In other words, the insertion sleeve can be movable relative to the shaft and attached to the shaft. Intuitive operation can be achieved in particular if the shaft has a linear degree of freedom and a rotational degree of freedom relative to the insertion sleeve - specifically, axial mobility in combination with rotation, but fixed position with respect to a radial position. In some embodiments, a rotational position can be fixed and/or definable. The resectoscope device may comprise a user-operated locking mechanism. In the locked position, only axial movement is possible. Alternatively or additionally, an axial position can also be locked. A locking mechanism, in particular the aforementioned locking mechanism, may also be provided for this purpose. In this case, only rotation can remain as a degree of freedom in the locked state. Particularly intuitive operation is achieved if the shaft is linearly movable and rotatable relative to the insertion sleeve.

In other words, the insertion sleeve can function like a trocar, specifically a captive trocar, as known from minimally invasive surgery. It can therefore provide trocar-like access to the cavity, in particular the bladder.

The longitudinal axis can run parallel to the main extension direction of the insertion sleeve. When said sleeve is at least partially inserted into the cavity, a longitudinal axis of the shaft may also run parallel to the main extension direction of the insertion sleeve and/or the longitudinal axis thereof.

The insertion sleeve can have a smaller longitudinal extension than the shaft. In some embodiments, the longitudinal extension of the insertion sleeve can, for example, be at most 95%, in particular at most 80%, preferably at most 70%, particularly preferably at most 50%, of the longitudinal extension of the shaft. Longitudinal extension refers to the extension along the longitudinal axis. The shaft can, for example, be between 20 cm and 40 cm long, in particular between 20 cm and 30 cm, preferably between 20 cm and 25 cm.

The insertion sleeve can have an annular, in particular circular, cross-section, in particular in a distal end piece. The wall thickness of the insertion sleeve can, for example, be 0.5 mm to 1.5 mm. For example, an outer diameter can be up to 10 mm, in particular up to 9 mm, preferably up to 8 mm. In principle, a small diameter is desirable in order to minimize the burden on the patient.

The insertion sleeve can be made of stainless steel, medical-grade plastic material, titanium, polyether ether ketone, and/or the like. In some embodiments, the insertion sleeve is intended for single use, or is a single-use product. The insertion sleeve may have a friction-reducing coating on its inner surface - for example, Teflon, silicone, and/or the like.

The resectoscope device can also comprise a proximal operating module that can be gripped by a user and by means of which the shaft can be manipulated. The user can therefore orient the tool tip inside the bladder by manipulating the operating module.

Furthermore, the entire operating module can be movable relative to the insertion sleeve. The insertion sleeve can therefore remain stationary and/or move only minimally when the proximal operating module is moved. If the insertion sleeve is inserted corresponding to the urethra, movement of the insertion sleeve relative to the urethra can be reduced, without restricting longitudinal mobility of the shaft and/or the resection tool. The shaft and/or the resection tool can, for example, be coupled proximally to the operating module. By moving the operating module back and forth, the user can move the shaft and the resection tool, in particular jointly, relative to the insertion sleeve and, in particular, perform an ablation movement.

The shaft and the resection tool can therefore be moved independently of each other, in particular by one user, with the movement being carried out relative to the insertion sleeve. For a resection, the user can insert the insertion sleeve together with the shaft and the resection tool into the urethra. This can also happen sequentially, for example, if the different modules are of different lengths. The term “together" can be understood to mean that the insertion can be a single process, and the user does not have to grasp the individual modules one after the other and advance them individually through the urethra. With the tool partially inserted into the urethra or cavity, the user can optionally advance the resection tool and/or the shaft further, in particular to explore the bladder and/or to perform a resection.

Referring to the above statements regarding the independent movability of the resection tool relative to the shaft, in some embodiments, the proximal operating module further comprises an actuating mechanism by means of which the resection tool can be moved along the longitudinal axis independently of the shaft. By actuating the actuating mechanism, the user can move the resection tool back and forth, in particular independently of the shaft and/or the insertion sleeve, in particular to perform an ablation movement.

In other words, the shaft and the resection tool can be configured to be moved within the insertion sleeve after being inserted into the cavity during the ablation of tissue. In contrast to a conventional resection, the ablation of tissue does not involve moving a module that is in contact with the urethra.

The insertion sleeve can be detachably coupled to the operating module. For example, the operating module together with the insertion sleeve can form a coupling mechanism configured to detachably connect the insertion sleeve to the operating module. If necessary, the insertion sleeve can be released - for example, after being inserted into the cavity together with the shaft and the resection tool. The insertion sleeve can also be mechanically recoupled to the operating module if required in order to remove the insertion sleeve together with the shaft and the resection tool from the cavity.

In accordance with the above explanations, the resectoscope device can have different positions, which can be defined by a different longitudinal position of the resection tool to the shaft and to the insertion sleeve and/or a different longitudinal position of the shaft to the resection tool and to the insertion sleeve.

As previously described, the insertion sleeve can be configured to rest atraumatically during tissue ablation. Patient comfort is increased and postoperative complications are reduced. It is understood that the insertion sleeve could move slightly relative to the ureter. However, this movement may be negligibly small, or significantly smaller than the relative movement between a shaft and the urethra during a conventional resection. In other words, the insertion sleeve can be configured to remain at least substantially stationary when the shaft is positioned within the cavity in relation to surrounding and, in particular, contacting tissue.

Furthermore, the resectoscope device can comprise a return for a rinsing fluid, wherein the return is formed between the insertion sleeve and the shaft. Tissue residues generated during the ablation process can be rinsed out of the bladder to reduce turbidity of the fluid, in particular the rinsing fluid, in the bladder and to ensure good visibility of the tissue to be removed. In some embodiments, some play may be provided between the insertion sleeve and the shaft, which may produce a gap. This can form the return. The outer diameter of the shaft can be approximately up to 10%, in particular up to 7%, preferably up to 4%, smaller than the inner diameter of the insertion sleeve. The resectoscope device can be connected proximally to a suction device, by means of which a suction pressure can be generated to draw the rinsing fluid out of the cavity, in particular the bladder.

Precise bending and positioning of the tool tip can be achieved if the joint is a mechanical rotary joint. Improved controllability and higher stability of the resectoscope device can therefore be achieved thereby. A mechanical rotary joint can be understood as a connecting device between two portions of the carrier arm, which connects them such that they can rotate relative to each other about a fixed axis of rotation.

Furthermore, the joint can be actuated by a user, in particular by means of the operating module. This can mean that the user can bend the bending portion by an actuation. For example, the resectoscope device could comprise an actuating mechanism for actuating the joint, such as a Bowden cable, a pull and/or push mechanism, a control cable, a push rod, and/or a pull wire. An actuating mechanism comprising a rack and pinion is also conceivable, wherein, for example, the pinion can be arranged on the bending portion. By means of a linear movement of the rack along the longitudinal axis of the carrier arm, the bending portion can be bendable accordingly. The actuating mechanism can extend at least partially within the carrier arm - for example, within the cavity of the carrier arm.

Particularly precise positioning of the tool tip can be achieved if the joint has a single degree of freedom. By means of the joint, the bending portion can be rotated, for example, about an axis of rotation that runs perpendicular to and/or through the longitudinal axis of the carrier arm. In other words, the axis of rotation of the joint can run transversely to the longitudinal direction of the distal end piece. In some embodiments, the resectoscope device comprises two carrier arms, wherein the axis of rotation in these embodiments runs perpendicular to the respective longitudinal axis of the two carrier arms. In particular, the axis of rotation is arranged such that the tool tip is bendable within the observation region.

### BRIEF DESCRIPTION OF THE DRAWINGS

The present disclosure will be described by way of example below with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included, but not a second object. However, a number and/or sequence of objects could also be derived using numerical words.

FIG. 1 is a schematic representation of a resectoscope device in a side view;

FIG. 2 is a schematic representation of the resectoscope device viewed from a distal position;

FIG. 3 is a perspectival schematic representation of the resectoscope device in a first position;

FIG. 4 is a perspectival schematic representation of the resectoscope device in a second position;

FIG. 5 is a schematic side view of a resectoscope device in a third position;

FIG. 6 is a schematic representation of a resectoscope comprising the resectoscope device, a control unit, and a display device;

FIG. 7 is a schematic representation of the resectoscope device in a side view;

FIG. 8 is a schematic representation of a partial cross-section of the resectoscope device;

FIG. 9 is a further schematic representation of the resectoscope device;

FIG. 10 is a further schematic representation of the resectoscope device;

FIG. 11 is a schematic representation of a distal portion of the resectoscope device in a side view;

FIG. 12 is a further schematic representation of the resectoscope device;

FIG. 13 is a further schematic representation of the resectoscope device;

FIG. 14 is a further schematic representation of the resectoscope device;

FIG. 15 is a further schematic representation of the resectoscope device;

FIG. 16 is a further schematic representation of the resectoscope device;

FIG. 17 is a further schematic representation of the resectoscope device; and

FIG. 18 is a further schematic representation of the resectoscope device.

### DETAILED DESCRIPTION OF PREFERRED EMBODIMENTS

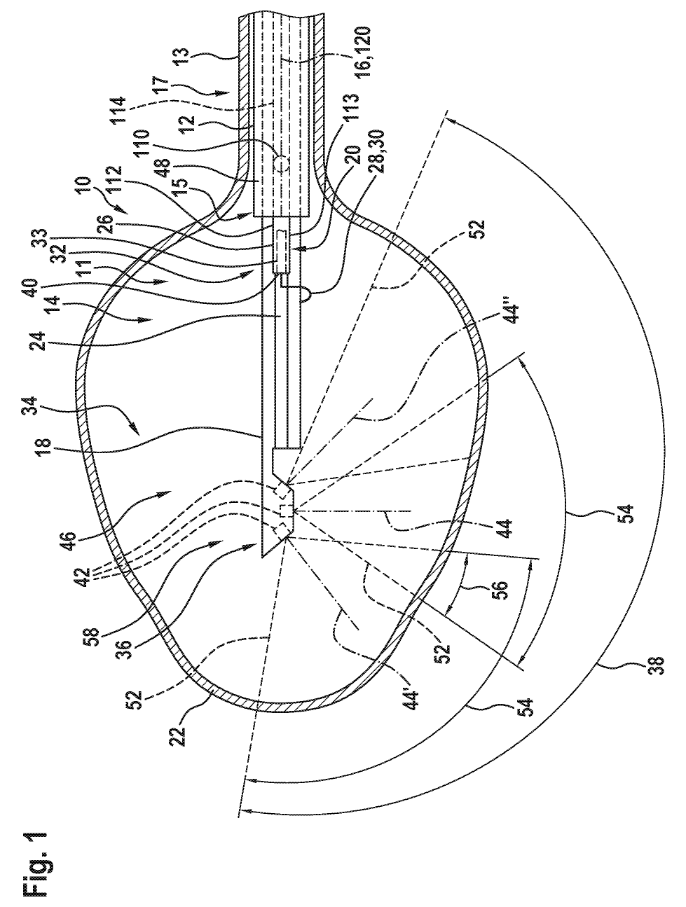

FIG. 1 is a schematic representation of a resectoscope device 10 in a side view. The resectoscope device 10 is configured to perform a resection and comprises an insertion sleeve 12 having a longitudinal axis 16, a shaft 18, and a resection tool 20. The resectoscope device 10 is shown in one of many possible positions. The positions are defined by a relative position of the insertion sleeve 12, the shaft 18, and the resection tool 20 to each other. These modules 12, 18, 20 can be moved independently of each other relative to each other.

The insertion sleeve 12 is shown in a state inserted into a urethra 13. A distal end piece 15 of the insertion sleeve 12 protrudes into a bladder 11. It is also conceivable that, in other situations, e.g., depending upon an anatomy of a patient, the insertion sleeve 12 does not extend into the bladder 11. Because the insertion sleeve 12 is tubular in shape and defines a guide channel 17, it provides trocar-like access to the bladder 11 for the shaft 18 and the resection tool 20.

The shaft 18 and the resection tool 20 extend along the longitudinal axis 16 of the insertion sleeve 12 through the guide channel 17 into the cavity 14, in this case the bladder 11. These modules 18, 20 can be inserted into the urethra 13 together with the insertion sleeve 12 by being mechanically coupled to each other. Once the insertion sleeve 12 is positioned in a desired position, the shaft 18 and the resection tool 20 can be advanced further distally.

The shaft 18 has a distal end piece 34, which is immovably connected to a main body 19 of the shaft 18. At the distal end piece 34, the shaft 18 has an image acquisition device 36 for endoscopic imaging of the interior of the bladder 11 and an illumination device 46 for illumination of the interior of the bladder 11. The image acquisition device 36 and the illumination device 46 are designed integrally together.

The image acquisition device 36 comprises three image acquisition units 42, which define different viewing directions 44, 44‘, 44‘‘. The image acquisition units 42 also each have a viewing angle 52. One image acquisition unit 42 has a viewing direction 44‘ toward the distal and another image acquisition unit 42 has a viewing direction 44‘‘ toward the proximal. The image acquisition units 42 together define a continuous observation region 38. Each of the image acquisition units 42 has its own observation sub-region 54 corresponding to the relevant viewing angle 52. Each of these has at least one overlap region 56 that partially overlaps an observation sub- region 54 of an adjacent image acquisition unit. The image acquisition units 42 each comprise an input optics and an image sensor for image generation (both not shown in detail), which in particular comprises a CCD chip or CMOS chip. The image acquisition units 42 are integrally designed with illumination units (not shown in detail) of the illumination device 46, which are designed as LED’s.

The resection tool 20 comprises at least one carrier arm 26 and a tool tip 28, which is designed as an HF resection loop. More precisely, the resection tool 20 comprises two carrier arms 26, as described below. A guide device 24 is formed on a surface of the shaft 18, which device is designed as a guide groove for the carrier arm 26. The guide device 24 extends along a large extent of the shaft 18 into the distal end piece 34. The end of the guide device 24 in the distal end piece 34 is provided as an open guide groove. The guide device 24, in particular the guide groove, partially incorporates the carrier arms 26 of the resection tool 20. As a result, the carrier arms 26 run linearly along the longitudinal axis 120 of the shaft 18 in the guide device 24. The longitudinal axis 120 is arranged coaxially with the longitudinal axis 16. The shaft 18 and the resection tool 20 are movable independently of each other along the longitudinal axis 120 of the shaft 18. The carrier arms 26 can therefore be moved linearly from proximal to distal and vice versa in the guide device 24. This displacement allows the tool tip 28 to be moved linearly relative to the shaft 18 and independently thereof. In particular, the tool tip 28 can be moved beyond a distal end 58 of the shaft 18 (see FIG. 4).

Furthermore, the resection tool 20, in particular the carrier arm 26, comprises a joint 110 by means of which the carrier arm 26 is bendable in an articulated manner. The carrier arm 26 forms at least part of the joint 110. More precisely, by means of the joint 110, a bending portion 112 is bendable in an articulated manner relative to a portion 114, proximal to the joint 110, of the carrier arm 26. The bending portion 112 comprises a bendable portion 113 of the carrier arm 26 and the tool tip 28. Accordingly, the carrier arm 26, in particular the bending portion 112, is bendable relative to the distal end piece 34 of the shaft 18 by means of the joint 110. The tool tip 28 can thereby be moved within the observation region 38. For the joint 110 and its function, reference is also made to the following figures.

The resectoscope device 20 also comprises a rinsing device 32. This comprises a supply line 33 and an outlet 40. The supply line 33 extends along a longitudinal axis of the carrier arm 18 and within the carrier arm 26 from proximal to distal across the joint 110 to the outlet 40. The outlet 40 is arranged on the carrier arm 26, in particular at a distal end of the carrier arm 26, in particular on the bendable portion 113 of the carrier arm 26. A rinsing fluid can be conveyed through the supply line 33 and exits at the outlet 40. The rinsing fluid can be used to fill the bladder 11 for resection and also to rinse away tissue residues that arise during the resection. The supply line 33 can be designed in some portions as a channel that is formed in the carrier arm 26. In some portions, the supply line 33 can also be formed by a flexible line such as a hose (not shown in detail). By means of the flexible line, the rinsing fluid can be conducted via the joint 110. In some embodiments, the flexible line runs in the cavity (not shown) of the carrier arm 26 over at least most of the longitudinal extent of the carrier arm 26.

Furthermore, the resectoscope device 10 comprises a proximal operating module (see FIG. 6), which is movable as a whole relative to the insertion sleeve 12. A user can hold the resectoscope device 10 on the operating module and operate a resectoscope comprising the resectoscope device 10 (see FIG. 6). The proximal operating module can be gripped by the user, and the shaft 18 can be manipulated by means of it.

Using the resectoscope device 10, a resection can be performed by the user as follows. First, he guides the shaft 18 and the resection tool 20 together with the insertion sleeve 12 into the cavity 14, in this case the urethra 13 and partly the bladder 11. He then brings the insertion sleeve 12 into a desired position within the urethra 13. Now, he fills the bladder 11 using the rinsing device 32 to open it up. He then positions the distal end piece 34 of the shaft 18 comprising the image acquisition device 36 within the bladder 11. To do this, he pushes the shaft 18 distally relative to the insertion sleeve 12. The insertion sleeve 12 remains in its original position. This reduces the total distance traveled by a relative movement between a resectoscope device and the urethra compared to the prior art.

During the advancement movement, the resection tool 20 is coupled to the shaft 18. This means that both modules 18 and 20 are advanced together. By pivoting the shaft 18 and, if necessary, advancing it, the user can gain an overview of the interior of the bladder 11 and examine the tissue 22 on an inner wall of the bladder 11. This allows him to identify tissue that needs to be ablated, such as tissue that shows a pathological change. An example of such tissue is cancerous tissue.

This identified tissue can then be ablated using the tool tip 28. To do this, the user brings the tool tip 28 into the vicinity of the tissue by bending the bending portion 112, pivoting the shaft 18 and longitudinally positioning the shaft 18. In the process, the user positions the distal end piece 34 such that the tissue to be ablated is arranged in the observation region 38 and can be clearly imaged. During longitudinal positioning, the insertion sleeve 12 remains stationary relative to the urethra 13 or the cavity 14, as already described. In other words, the insertion sleeve 12 rests atraumatically.

According to one embodiment, after positioning the distal end piece 34 and selecting an angular position of the bending portion 112 (see the following figures), the user moves the resection tool 20 relative to the shaft 18, wherein the insertion sleeve 12 also remains stationary and rests atraumatically, and brings the tool tip 28 into contact with the tissue. After activation of the tool tip 28, if it is designed as an HF resection loop, for instance, the tissue can be ablated layer by layer. For this purpose, the tool tip 28 is iteratively swept over the tissue, thereby superficially treating and ablating the tissue to be ablated. To sweep over the tissue, the resection tool 20 can be moved relative to the shaft 18. Furthermore, the shaft 18 can be moved together with the resection tool 20, while the insertion sleeve 12 remains stationary in the urethra 13. The angular position is maintained during the movement.

According to another embodiment, the tissue ablation movement is carried out by bending the bending portion 112. The shaft 18 can be held almost stationary in the process. For more detailed explanations of both embodiments, reference is made to the following figures.

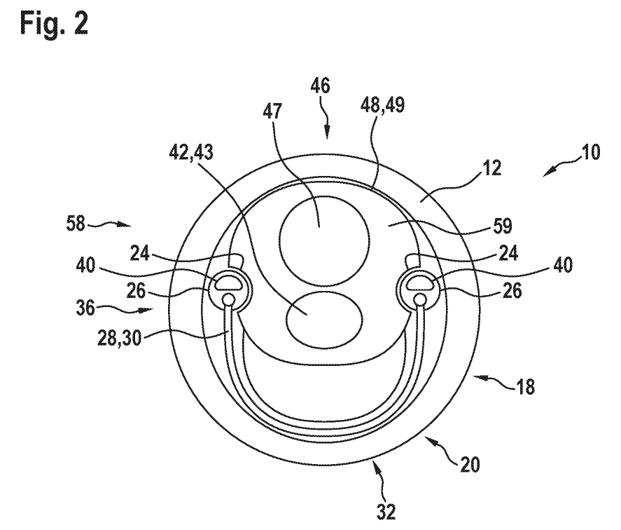

During tissue ablation, rinsing is carried out constantly by means of the rinsing device 32, and rinsing fluid from the bladder 11 is conveyed out of the bladder 11 by means of a return 48 (see FIG. 2).

After resection is completed, the shaft 18 can be pulled out of the cavity 14 together with the resection tool 20 and the insertion sleeve 12.

The shaft 18 and the resection tool 20 are therefore configured to be moved within the insertion sleeve 12 after being inserted into the cavity 14 during the ablation of tissue 22. The insertion sleeve 12 is configured to rest atraumatically during the ablation of tissue 22.

FIG. 2 shows a schematic representation of the resectoscope device 10 in a view from distal to a distal end 58 of the shaft 18 or to a distal end face of the resectoscope device 10. A distal end face 59 of the shaft 18 can be seen on which an illumination unit 47 of the illumination device 56 is arranged, which is designed as an LED. Input optics 43 of one of the image acquisition units 42 is also arranged on the distal end face 59.

In addition, the guide device 24 can be seen, the distal end of which is laterally embedded in the distal end face 59. The carrier arms 26 are guided in the guide device 24. The resection tool 20 comprises two carrier arms 26. The tool tip 28, in particular the HF resection loop 30, is arranged between the carrier arms 26. The shaft 18 is arranged between the carrier arms 26. Furthermore, the outlet 40 of the rinsing device 32 of the carrier arm 26 can be seen. Both carrier arms 26 are identical in design and correspondingly each have a outlet 40.

The shaft 18 has play in the insertion sleeve 12. Therefore, a gap 49 can be seen between the insertion sleeve 12 and the shaft 18. This gap 49 forms the return 48. The rinsing fluid can therefore be extracted through the gap 49.

In the illustrated exemplary embodiment, the insertion sleeve 12 has an outer diameter of 9 mm and an inner diameter of 7.5 mm. The shaft 18 has an outer diameter of 6.5 mm. The shaft 18 has a length of 30 cm. The insertion sleeve 12 has a length of 25 cm. It is also conceivable to use significantly shorter insertion sleeves 12, which are, for example, half as long as the shaft 18. Even shorter insertion sleeves 12 are also conceivable. The dimensions mentioned are merely examples.

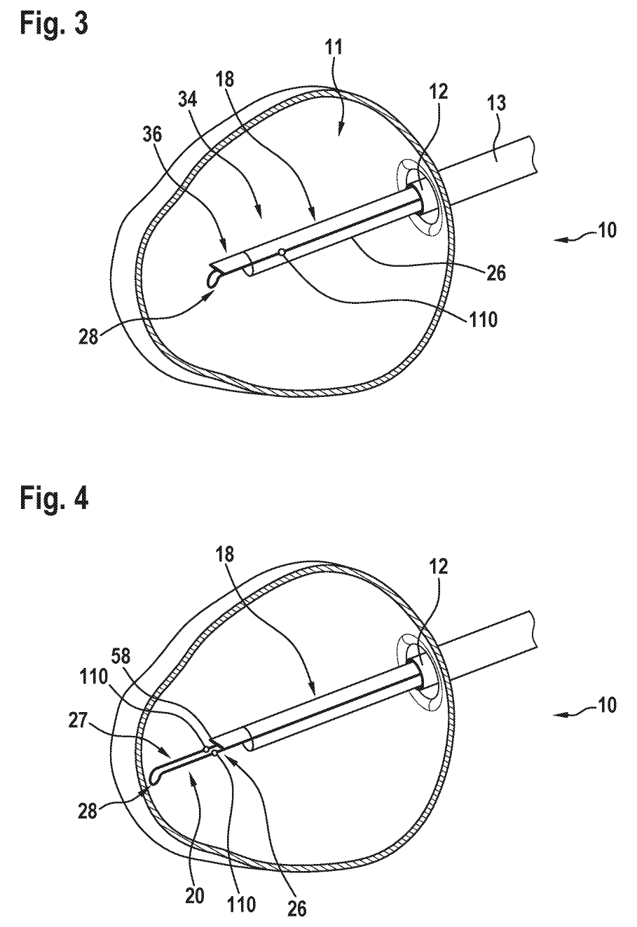

FIGS. 3 and 4 each show a perspectival schematic representation of the resectoscope device 10 in different positions. These are defined by a different relative position of the resection tool 20 with respect to the shaft 18.

In FIG. 3, the resection device 10 with the image acquisition device 36 can be seen in the distal end piece 34 of the shaft 18. Furthermore, the resection tool 20 can be seen together with the tool tip 28 and one of the carrier arms 26 and the joint 110. In addition, the insertion sleeve 12, which is inserted into the urethra 13, can be seen. The shaft 18 and the resection tool 20 extend through the insertion sleeve 12 and into the bladder 11. The shaft 18 and the resection tool 20 can be moved together relative to the insertion sleeve 12, with the insertion sleeve 12 remaining stationary in the urethra 13. In the position shown in FIG. 3, the tool tip 28 is arranged flush with the distal end 58 of the shaft 18.

As can be seen in FIG. 4, the resection tool 20 can be moved independently of the shaft 18 relative thereto and to the insertion sleeve 12. In the position shown in FIG. 4, the tool tip 28 is arranged distally beyond the distal end 58 of the shaft 18. The insertion sleeve 12 remained in the same position as shown according to FIG. 3. The resection tool 20 was therefore moved independently of the shaft 18. In addition, a further carrier arm 27 can be seen. As described above, this is identical in design to the carrier arm 26. Accordingly, it is also bendable. Furthermore, one joint 110 per carrier arm 26, 27 can be seen. The tool tip 28 is arranged between the carrier arm 26 and the further carrier arm 27. Referring to the previous figures, the shaft 118 extends in the angular position shown between the bendable portions 113 of the carrier arms 26, 27. This configuration is referred to as the insertion configuration. Since the carrier arms 26, 27 are not bent, the resection tool 20 can be introduced into the body cavity via the insertion sleeve 12. Accordingly, the angular position shown is an insertion angular position. In this, the bending portion 112 extends parallel to the longitudinal axes 16, 120.

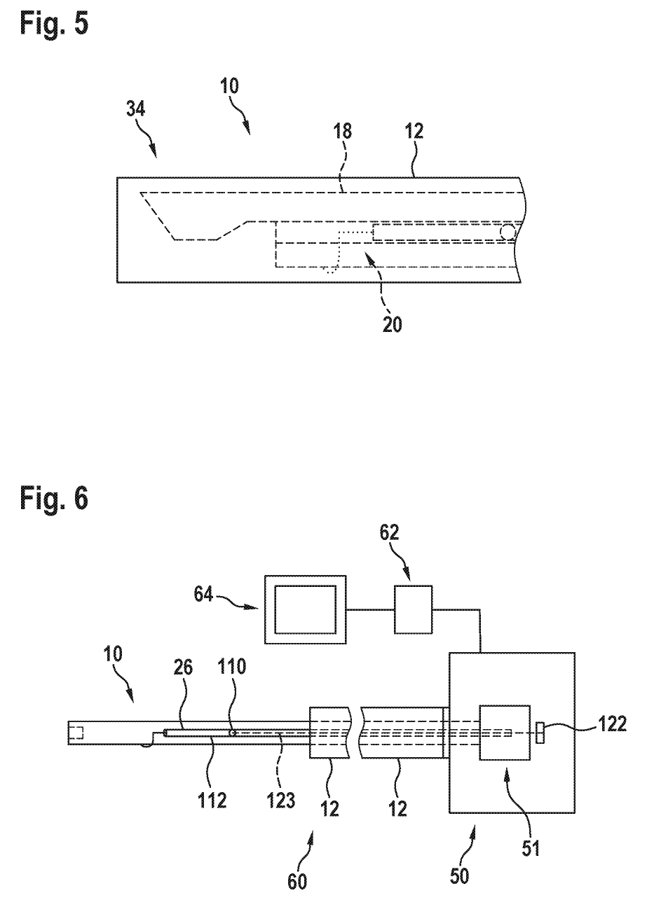

FIG. 5 shows a schematic side view of the resectoscope device 10 in another position. In the position shown, the distal end piece 34 of the shaft 18 is completely enclosed by the insertion sleeve 12. The resection tool 20 also does not extend distally beyond the insertion sleeve 12. In the position shown, the resectoscope device 10 could, for example, be inserted guided into the cavity 14. However, other positions are also conceivable in which at least one of the modules 18, 20 extends distally beyond the insertion sleeve 12.

FIG. 6 shows a schematic representation of the resectoscope 60, which comprises the resectoscope device 10. Furthermore, the proximal operating module 50 can be seen and is movable as a whole relative to the insertion sleeve 12. The proximal operating module 50 also comprises an actuating device 51 by means of which the resection tool 20 can be operated, in particular moved forwards and backwards. Furthermore, the operating module 50 forms at least part of an actuating device 122 for actuating the joint 110. The actuating device is not shown in detail. It is conceivable, for example, to use a rack that extends approximately along the line 123 within the carrier arm 26 to the joint 110. A pinion, which engages with the rack, can correspondingly be formed on the bending portion 112. The user can therefore bend the bending portion 112 by actuating the rack.

The resectoscope 60 is connected to a control unit 62, which can, for example, process image data and control a function of the resectoscope 60. In particular, the image acquisition device can be controlled by means of the control unit 62. The control unit 62 is connected to a display device 64, on which a representation of an endoscopic image can be generated. The representation allows the user to view the inside of the bladder 11 and thus monitor the resection procedure.

The operating principle and features of the resectoscope device 10 are explained in more detail using the following figures.

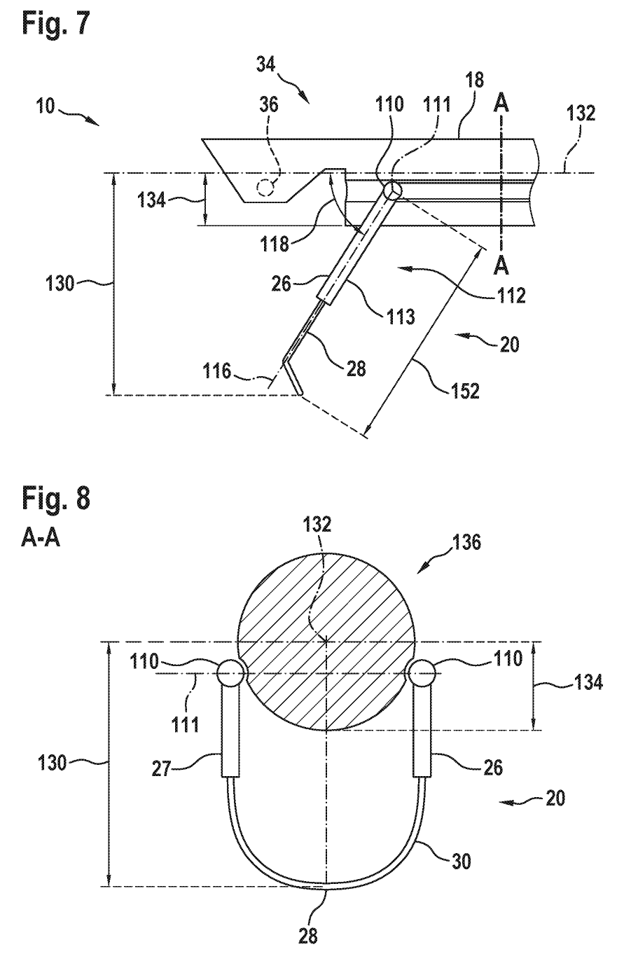

FIG. 7 and FIG. 8 show various schematic representations of the resectoscope device 10. FIG. 7 shows it in a side view, and FIG. 8 shows a partial cross-section of the resectoscope device 10. The resectoscope device 10 comprises the shaft 18, the image acquisition device 36 (see FIG. 1 to FIG. 6), which is arranged on the distal end piece 34 of the shaft 18, and the resection tool 20. This has the tool tip 28, the carrier arm 26, and the joint 110. The resection tool 20 is bendable in some portions by means of the joint 110. Specifically, the bending portion 112 is bendable in relation to the portion, arranged proximally to the joint 110, of the carrier arm 26 and can be moved into an angular position by bending. As already explained, the bending portion 112 comprises the bendable portion 113 of the carrier arm 26 and the tool tip 28. As can be seen in FIG. 8, the resectoscope device comprises two carrier arms 26, 27, between which the tool tip 28 extends.

In FIG. 7 and FIG. 8, the resection tool 20 is shown in an angular position. In this, the bending portion 112 is bent in relation to the longitudinal axis 132 of the distal end piece 34. The carrier arm 26, in particular the bendable portion 113 of the carrier arm 26, is therefore bendable relative to the distal end piece 34 of the shaft 18. In the angular position of the carrier arm 26 shown in FIG. 7, the tool tip 28 is at a distance 130 from the longitudinal axis 132 of the distal end piece 34. The distance 130 describes the distance between the point of the bending portion 112 furthest away from the longitudinal axis 132, and the longitudinal axis 132. This distance 130 is larger than a radius 134 of a cross-sectional profile 136 of the distal end piece 34 by a factor of approximately 4 (see also FIG. 8). In the case shown, the radius 134 is a largest radius of the distal end piece 34, wherein a portion of the shaft 18 that can be arranged within the body cavity or bladder during resection defines the distal end piece 34. FIG. 8 shows an angular position in which the distance 130 is twice as large as the radius 134.

Furthermore, a length 152 of the bending portion 112 can be seen in FIG. 7. This is measured as a distance between the joint 110 and a point of the bending portion 112 furthest away from the joint 110, wherein the distance is measured parallel to the principal extension direction of the bendable portion 113. The length 152 of the bending portion 112 exceeds the radius 134 of the cross-sectional profile 136 of the distal end piece 34 by a factor of approximately 3.5. More precisely, the length 152 of the bending portion 112 is 25 mm.

The joint 110 is a mechanical rotary joint and has a single degree of freedom. This is a rotational degree of freedom. An axis of rotation 111 of the joint 110 runs orthogonally to the longitudinal axis 132 of the distal end piece 34 (see FIG. 7 and FIG. 8). As can be seen in FIG. 8, the axis of rotation 111 runs on an imaginary connecting line between the respective joints 110 of the carrier arms 26, 27. The carrier arms 26, 27 therefore each have a joint 110, which defines an axis of rotation 111 in each case. The axes of rotation 111 of the joints 110 are arranged coaxially.

By means of the joint 110, the bendable portion 113 can be moved into an angular position in which a main extension axis of the bendable portion 113 and the longitudinal axis 132 enclose an arm angle 118 of at least 45°. In some embodiments, the joint allows an arm angle 118 of at least 90°, in particular at least 135°, and/or an arm angle 118 of up to, for example, 170°, in particular 175°.

As explained in more detail below, the angular positions of the carrier arm 26 shown in FIG. 7 and FIG. 8 each define an application configuration in which a resection is performed.

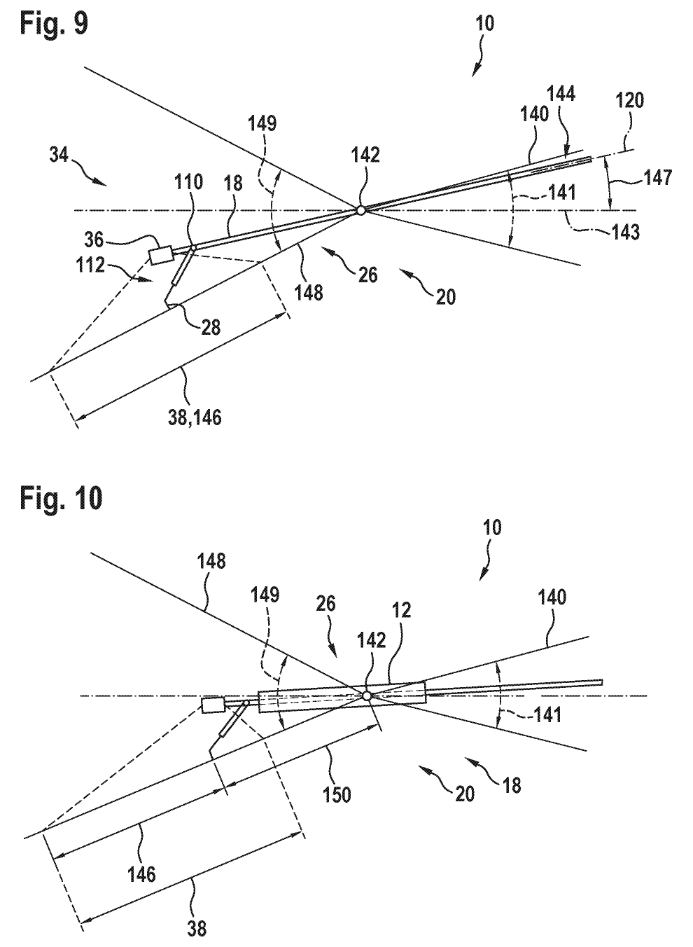

FIG. 9 shows the resectoscope device 10 in a further schematic representation. The angular position of the carrier arm 26 corresponds to the angular position according to FIG. 7. This was set by selection of the angular position. The bending portion 112 was therefore bent relative to the distal end piece 34. In this angular position, the tool tip 28 is movable on a surface of an application cone 148. To move on the surface of the application cone 148, the user can move a proximal portion 144 of the shaft 18 within a manipulation cone 140. This is achieved by pivoting about a pivot point 142 and by linearly displacing the shaft 18 through the pivot point 142. If, for instance, the user moves the tool tip 28 closer to the pivot point 142, the user pivots the shaft 18 such that an angle 147 between the longitudinal axis 120 of the shaft 18 and a central axis 143 of the manipulation cone 140 becomes smaller. In the opposite direction, the user increases the angle 147 accordingly. A maximum distance of the tool tip 28 from the pivot point 142 on the surface of the application cone 148 is accordingly limited by an aperture angle 141 of the manipulation cone 140 when the angular position of the carrier arm 26 is selected. Since the resection tool 20 together with the shaft 18 can be rotated about the longitudinal axis 120 of the shaft 18, the application cone 148 and the manipulation cone 140 have the shape of a circular cone. Accordingly, the aperture angle 141 corresponds to twice the angle 147.

Furthermore, the image acquisition device 36 in the distal end piece 34 defines the observation region 38, as described above. This intersects a region of the application cone 148. The tool tip is movable within this region - a surface portion 146 of the application cone 148. More precisely, the tool tip 28 can be moved by manipulating the proximal portion 144 of the shaft 18 within the manipulation cone 140 and selecting the angular position of the carrier arm 26 on the surface portion 146, located within the observation region 38, of the application cone 148. In the case shown, the surface portion 146 is defined by the observation region 38. This means that the tool tip 28 could also be movable outside the observation region 38.

Due to the angular position of the carrier arm 26, the aperture angle 149 of the application cone 148 is larger than the aperture angle 141 of the manipulation cone 140. Since the tool tip 28 was bent beyond the cross-section of the shaft 18, tissue further away can be reached with a smaller pivot of the shaft 18 in comparison with a non-bendable carrier arm (not shown). The aperture angle 149 of the application cone 148 is at least 1.5 times larger than the aperture angle 141 of the manipulation cone 140 - in the case shown, at least 2 times larger.

In FIG. 10, the resectoscope device 10 is shown in a schematic representation wherein the resectoscope device 10 is arranged in a position other than in FIG. 8. In addition, the insertion sleeve 12 can be seen, through which the shaft 18 and the resection tool 20 extend. The manipulation cone 140, the application cone 148, and the aperture angles 141, 149 correspond to those shown in FIG. 9. The angular position is also the same. It can be seen that the pivot point 142 is located inside the insertion sleeve 12. Due to the angular position of the carrier arm 26, the resection tool 20 cannot be pulled back further proximally. Accordingly, the traceable surface portion 146 is limited by the insertion sleeve 12 and the choice of angular position. In the embodiment shown, the distance 150 of the surface portion 146 of the application cone 148 from the pivot point 142 is at least 2 times larger than the length 152 of the bending portion 112 (see FIG. 7). The observation region 38 therefore extends proximally beyond the surface portion 146.

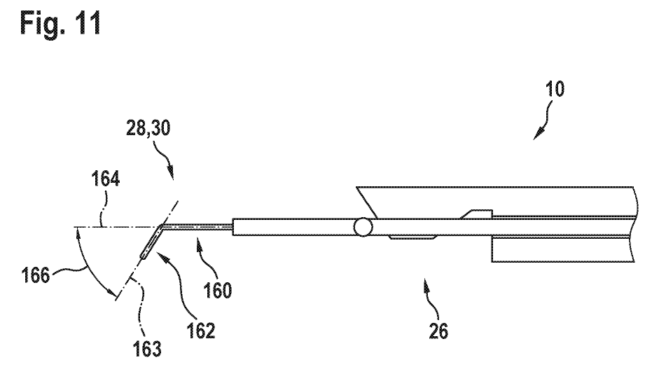

FIG. 11 is a schematic representation of a distal portion of the resectoscope device 10 in a side view. The carrier arm 26 is in the insertion configuration. The tool tip 28 is designed as an HF resection loop 30, as can be seen in more detail in FIG. 8, for example. The HF resection loop 30 comprises a supply line portion 160 and a working portion 162. The working portion 162 is brought into contact with the tissue during resection for tissue ablation. The supply line portion 160 connects the working portion 162 to the carrier arm 26. The working portion 162, in particular a longitudinal axis 163 of the working portion 162, is bent relative to a longitudinal axis 164 of the supply line portion 160 by a loop angle 166 of at most 90°. In the embodiment shown, the loop angle 166 is 45°. The working portion 162 therefore extends partially distally from the supply line portion 160. This allows easy access to a tissue site of the bladder opposite the bladder neck in the shown insertion configuration. Since the carrier arm 26 is bendable, however, other tissue sites can also be easily reached, and the tool tip 28 can be arranged in an application-specific manner by selecting the angular position.

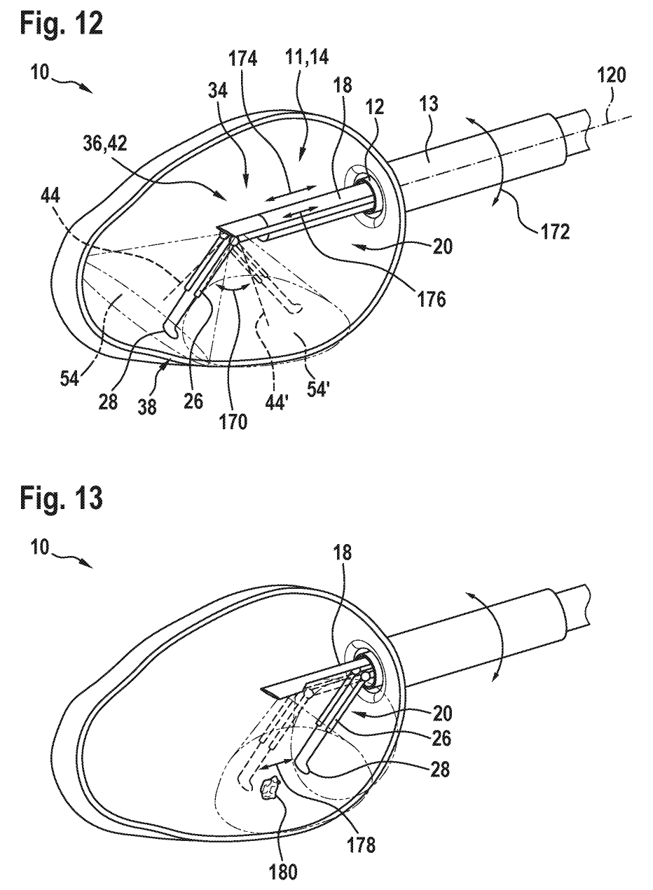

FIG. 12 is a further schematic representation of the resectoscope device 10. This is shown in a state partially inserted into the cavity 14 - in the illustrated case, the bladder 11. The shaft 18 and the resection tool 20 extend through the insertion sleeve 12, which is arranged in the urethra 13, and the distal end piece 34 with the image acquisition device 36 projects into the bladder 11. The resection tool 20 is shown in two different angular positions. Both define an application configuration. In the two different angular positions, the tool tip 28 is arranged in different observation sub-regions 54, 54'. The observation sub-regions 54, 54' are each assigned to different image acquisition units 42 of the image acquisition device 36, wherein the image acquisition units 42 define different viewing directions 44, 44' (see also FIG. 1). In the first angular position (solid lines), the tool tip 28 is arranged in a partially distally directed observation sub-region 54. In the second angular position (dashed lines), the tool tip 28 is arranged in an observation sub-region 54' that is assigned to an image acquisition unit 42 with a viewing direction 44' arranged perpendicular to the longitudinal axis 120 of the shaft 18. The tool tip 28 can be moved from the first observation sub-region 54 to the second observation sub-region 54' by bending the carrier arm 26 or by selecting an angular position. The tool tip 28 can therefore be moved by the bending of the carrier arm 26 between the observation sub-regions 54, 54'.

The user can therefore position the tool tip 28 in the direction of the arrow 170 by bending the carrier arm 26. Furthermore, the user can position the tool tip 28 in the direction of the arrow 172 by pivoting the shaft 18 and in the direction of the arrow 176 by pushing the resection tool 20 forwards. The user can position the shaft 18 in the direction of the arrow 174 - for example, to orient the image acquisition device 36. Furthermore, the user can rotate the shaft 18 together with the resection tool 20 about the longitudinal axis 120 (not shown).

It is explained below, with reference to FIGS. 13 to 18, how the user can perform a resection using the resection tool 20 with bendable carrier arm 26. Basically, two tissue ablation movements are conceivable (not an exhaustive list). On the one hand, the tissue could be ablated primarily by longitudinal displacement of the resection tool 20, together with the shaft 18 or independently of it (FIG. 13). During this tissue ablation movement, an angular position of the carrier arm 26 is selected once before the movement is performed, and then maintained. The tissue ablation movement thus corresponds approximately to that performed in a conventional resection. In contrast, the shaft 18 does not need to be pivoted as much, since, by choosing the angular position, it is possible to reach remote tissue sites with minimal pivoting of the shaft 18.

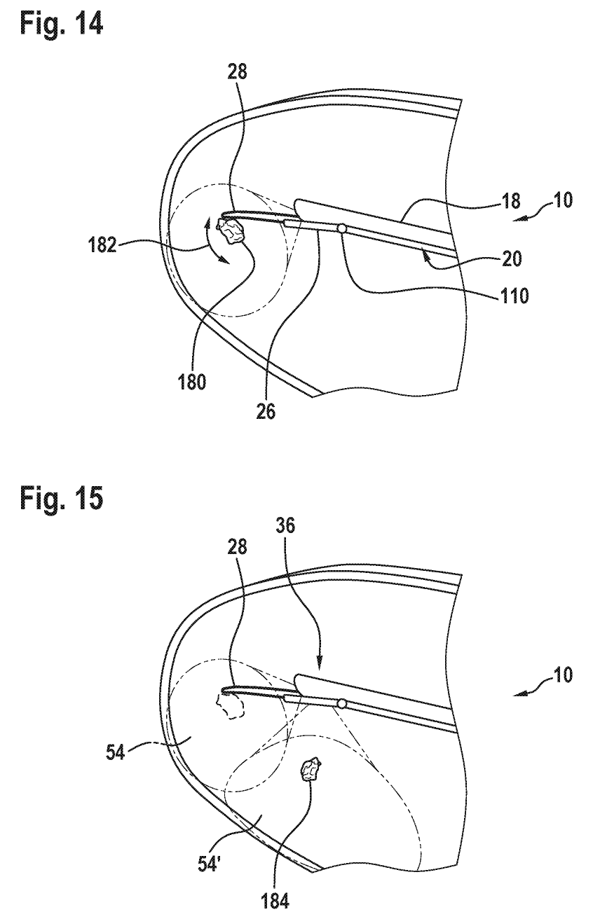

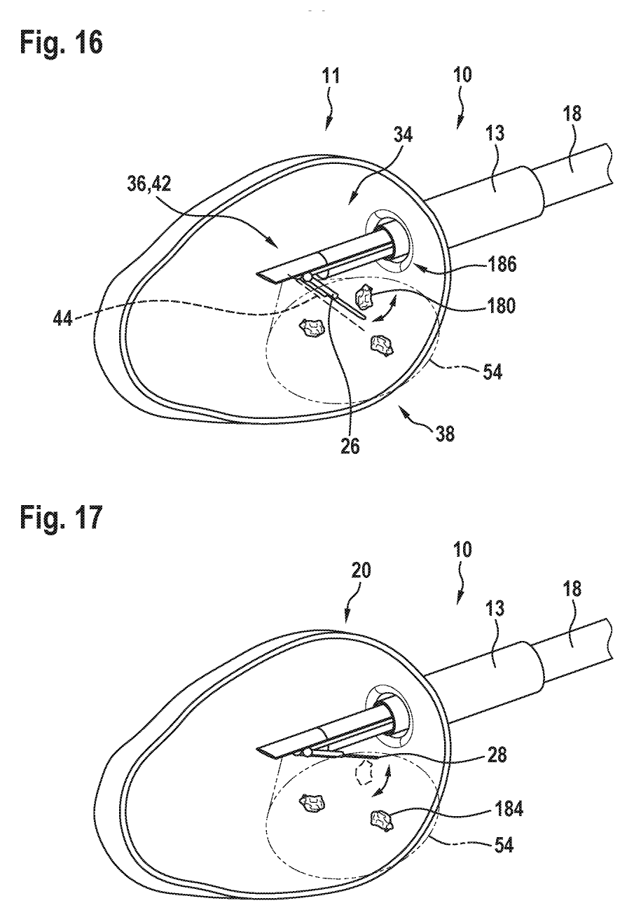

On the other hand, the tissue could be ablated by a looping movement of the bending portion 112 (FIG. 14 and FIG. 16). During the looping movement, the carrier arm 26 is bent continuously. In this case, the tissue ablation movement is therefore a rotational movement of the carrier arm 26 and not a linear movement of the resection tool 20.

A combination of both tissue ablation movements is also conceivable. For example, a first tissue site could be treated by means of the linear movement first, and then a second tissue site could be treated by means of the looping movement.

FIG. 13 shows a further schematic representation of the resectoscope device 10, wherein two different linear positions of the resection tool 20 are shown. These linear positions are achieved by a relative displacement of the resection tool 20 relative to the shaft 18. The angular position of the carrier arm 26 remains unchanged in both linear positions. Moving between the two linear positions results in a movement of the tool tip 28 along the direction of the arrow 178. In this way, the tool tip 28 can be iteratively moved over tissue 180 to be removed, and this tissue can be removed layer by layer.

FIG. 14 is a further schematic representation of the resectoscope device 10. The looping movement is carried out by continuously changing the angular position of the carrier arm 26. During this time, the resection tool 20 proximal to the joint 110 and the shaft 18 remain stationary relative to each other. The continuous bending means that the tool tip 28 moves iteratively over the tissue 180 to be removed along the direction of the arrow 182. Layer-by-layer tissue ablation is correspondingly made possible by the looping movement.

FIG. 15 shows a further schematic representation of the resectoscope device 10, wherein the tissue 180 has been removed according to FIG. 14. Since the image acquisition device 36 defines the multiple observation sub-regions 54, a large part of the bladder interior can be monitored without the need to reposition the image acquisition device 36. As shown, there can be further tissue 184 to be removed in the observation sub-region 54', for instance. This can be identified by the user without moving the image acquisition device 36 after the removal of the tissue 180 (see FIG. 14). According to the explanations, e.g., regarding FIG. 12, the tool tip 28 can correspondingly be moved into the observation sub-region 54'. Here, the tissue 184 can then be ablated either by means of the linear movement or the looping movement.

FIG. 16 is a further schematic representation of the resectoscope device 10. The image acquisition device 36 comprises an image acquisition unit 42 with a viewing direction 44 that is directed partially proximally. For the purpose of performing the resection, the distal end piece 34 of the shaft 18 can thereby be arranged within a bladder 11 such that the bladder neck 186 is located within the observation region 38 of the image acquisition device 36. Accordingly, a resection can also be performed in the vicinity of the bladder neck 186 or the urethra 13. This represents a particularly challenging resection. Nevertheless, this can be performed gently for the patient by using the looping movement of the carrier arm 26 for tissue ablation. Extensive pivoting of the shaft 18 is not necessary.

After tissue ablation, further tissue 184 to be removed can be identified in the observation region 54, as shown in FIG. 17, which shows a further schematic representation of the resectoscope device 10. By rotating the shaft 18 together with the resection tool 20, the tool tip 28 can be reoriented to reach the further tissue 184 to be removed.

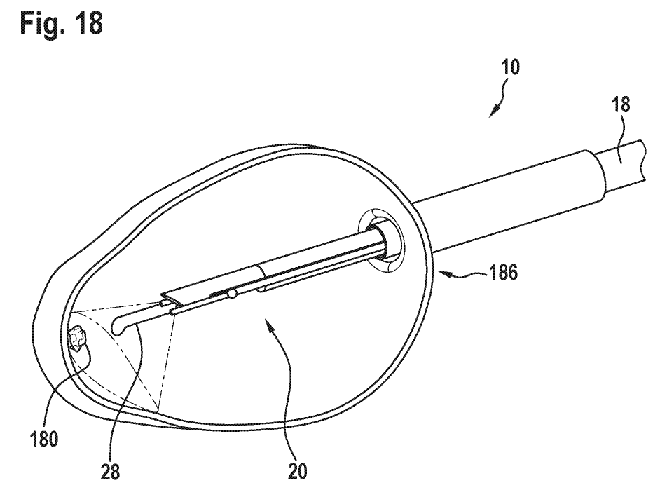

FIG. 18 is a further schematic representation of the resectoscope device 10. The resection tool 20 is shown in the insertion configuration. Due to the loop angle 166 of at most 90° (see FIG. 11 and associated description), tissue 180 to be removed that is opposite the bladder neck 186 can be reached in the insertion configuration. For this purpose, the shaft 18 does not need to be pivoted to a great extent due to the loop angle 166.

1. A resectoscope device, comprising:
a shaft;
an image acquisition device, which is located on a distal end piece of the shaft and defines an observation region;
a resection tool with a tool tip and a carrier arm; and
a joint by means of which the carrier arm is bendable in an articulated manner;
wherein, in at least one angular position of the carrier arm, a distance between the tool tip and a longitudinal axis of the distal end piece exceeds a radius of a cross-sectional profile of the distal end piece by at least a factor of 2.

2. The resectoscope device as set forth in claim 1, wherein the shaft defines a manipulation cone within which a portion, proximal to a pivot point, of the shaft can be manipulated by a user,
wherein the tool tip can be moved by manipulating the proximal portion of the shaft within the manipulation cone and selecting at least one angular position of the carrier arm on a surface portion, located within the observation region, of an application cone,
wherein an aperture angle of the application cone is at least 1.5 times as large as an aperture angle of the manipulation cone.

3. The resectoscope device as set forth in claim 2,
wherein the resection tool defines a bending portion that is bendable by means of the joint, and
wherein a distance of the surface portion of the application cone from the pivot point is at least 2 times larger than the length of the bending portion.

4. The resectoscope device as set forth in claim 1, wherein the image acquisition device includes at least two image acquisition units directed in different viewing directions, wherein the image acquisition units each define an observation sub-region of the observation region, and
wherein the tool tip can be moved between the observation sub-regions by bending the carrier arm.

5. The resectoscope device as set forth in claim 1, wherein the carrier arm is bendable relative to the distal end piece of the shaft by means of the joint, and
wherein the distal end piece is immovable relative to a main body of the shaft.

6. The resectoscope device as set forth in claim 1, wherein the shaft and the resection tool are movable independently of each other along a longitudinal axis of the shaft.

7. The resectoscope device as set forth in claim 1, wherein the resection tool defines a bending portion that is bendable by means of the joint, and
wherein a length of the bending portion exceeds a radius of a cross-sectional profile of the distal end piece by at least a factor of 3.

8. The resectoscope device as set forth in claim 7, wherein the length of the bending portion is in one of a range between 15 mm and 50 mm and 25 mm and 35 mm.

9. The resectoscope device as set forth in claim 1, further including a rinsing device by means of which a region that can be treated by the resection tool can be rinsed, comprising a supply line by means of which a rinsing fluid can be conducted along the shaft , wherein the supply line extends within the carrier arm .

10. The resectoscope device as set forth in claim 9, wherein the rinsing device comprises an outlet that is arranged on a portion, bendable by means of the joint, of the carrier arm.

11. The resectoscope device as set forth in claim 1, wherein the resection tool includes a further bendable carrier arm,
wherein the tool tip is arranged between the carrier arm and the further carrier arm, and
wherein, at least in an insertion configuration, the shaft extends between the bendable portions of the carrier arms.

12. The resectoscope device as set forth in claim 1, wherein the tool tip includes an HF resection loop having a supply line portion and a working portion, wherein the working portion is bent relative to a longitudinal axis of the supply line portion by a loop angle of at most 90°, and
wherein the working portion extends partially distally from the supply line portion.

13. The resectoscope device as set forth in claim 1, wherein, for the purpose of performing a resection, the distal end piece of the shaft is configured to be arranged at least partially within a bladder wherein the bladder neck is located within the observation region of the image acquisition device.

14. The resectoscope device as set forth in claim 1, wherein the resection tool together with the shaft is rotatable about a longitudinal axis of the shaft.

15. The resectoscope device as set forth in claim 1, further including an insertion sleeve configured to be partially inserted into a cavity of a patient and defines a longitudinal axis,
wherein the shaft extends within the insertion sleeve,
wherein the shaft and the resection tool are configured to be moved independently of each other relative to the insertion sleeve along the longitudinal axis of the insertion sleeve, and
wherein the shaft and the resection tool are configured to be inserted into the cavity together with the insertion sleeve

16. The resectoscope device as set forth in claim 1, further including a proximal operating module configured to be gripped by a user and by means of which the shaft is manipulated.

17. The resectoscope device as set forth in claim 1, wherein the joint is a mechanical rotary joint.

18. The resectoscope device as set forth in claim 1, wherein the joint has a single degree of freedom.

19. A resection tool comprising:
the resectoscope device as set forth in claim 1;
wherein the resection tool defines a bending portion that is bendable by means of the joint, and wherein the length of the bending portion is in one of a range between 15 mm and 50 mm and 25 mm and 35 mm.

20. A resectoscope having a resectoscope device as set forth in claim 1.
